(19) United States
(12) Patent Application Publication
(10) Pub. No.: US 2010/0003706 A1
(43) Pub. Date: Jan. 7, 2010

(54) SYPHILIS DIAGNOSTIC TESTS AND KITS

(75) Inventors: Wesley C. Van Voorhis, Seattle, WA (US); Caroline E. Cameron, Victoria (CA); Sheila A. Lukehart, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: WASHINGTON, UNIVERSITY OF, Seattle, WA (US); University of Victoria Innovation and Development Corporation, Victoria (CA)

(21) Appl. No.: 12/491,715

(22) Filed: Jun. 25, 2009

Related U.S. Application Data
(63) Continuation-in-part of application No. 11/107,373, filed on Apr. 14, 2005.

(51) Int. Cl. G01N 33/571; C07K 14/195; C12N 15/74; C12N 1/21

(52) U.S. Cl. 435/7.36; 530/350; 435/320.1; 435/252.3

(57) ABSTRACT

The present invention provides methods and kits for determining whether a human subject is infected with T. pallidum and isolated polypeptide fragments of T. pallidum proteins Tp92, Tp0453, and Gpd that can be used in the methods and kits of the invention.

### CROSS-REFERENCE TO RELATED APPLICATION

This application is a continuation-in-part application of U.S. patent application Ser. No. 11/107,373, filed Apr. 14, 2005, which is hereby incorporated by reference herein.

### STATEMENT OF GOVERNMENT LICENSE RIGHTS

This invention was made with government support under grant numbers R01 AI43456 and R01 AI 51334, awarded by the National Institutes of Health. The government has certain rights in the invention.

### FIELD OF THE INVENTION

The present invention relates to methods for diagnosing syphilis in a test subject.

### BACKGROUND OF THE INVENTION

The spirochetes are an order of distinctive bacteria that has long, helically coiled, cells. They are distinguished by the presence of flagella, called axial filaments, which run lengthwise between the cell membrane and cell wall. These cause a twisting motion that allows the spirochete to move about. Most spirochetes are free-living and anaerobic.

Treponema pallidum, subspecies pallidum, (hereinafter referred to as Treponema pallidum or T. pallidum) is a spirochete that causes syphilis in human beings. T. pallidum enters the host via breaches in squamous or columnar epithelium and primary infection is normally via sexual contact. T. pallidum can be transmitted to a fetus by transplacental passage during pregnancy.

Syphilis is characterized by primary, secondary, and tertiary clinical stages. The primary stage involves multiplication of the bacteria at the site of entry to produce a localized infection. The secondary stage occurs following an asymptomatic period and involves dissemination of the bacteria to other tissues. The tertiary stage may not occur for many years after infection and can cause damage to the brain and central nervous system and ultimately lead to death.

Syphilis diagnosis during the early primary stage can be accomplished by dark-field microscopy of a sample of primary chancre to identify the presence of spirochetes. Following the resolution of the primary chancre and in clinics lacking dark-field microscopy, the mainstay of syphilis diagnosis is a variety of serologic tests. The most common screening tests are the rapid plasma reagin (RPR) and Venereal Disease Research Laboratory (VDRL) tests, both of which test for the presence of antilipoidal antibodies. The antilipoidal antibodies recognize lipid material released from damaged host cells and from T. pallidum. Because neither of these tests uses syphilis-specific antibodies, there are problems associated with both their specificity and their sensitivity. In early primary disease, antilipoidal antibodies may not have developed and in late syphilis, up to thirty percent of individuals may lack antilipoidal antibodies. In addition, because a variety of conditions (e.g., lupus and increased age) lead to antilipoidal antibodies and false-positive results, a confirmatory test is often required.

Confirmatory tests include FTA-Abs (fluorescent treponemal antibody absorption test), MHA-TP (micro-hemagglutination assay for T. pallidum), TPPA (Treponema pallidum particle agglutination assay) (Pope, V., et al., J. Clin. Microbiol. 38:2543-5, 2000), and TPHA (T. pallidum hemagglutination assay), which use crude T. pallidum antigens (Larsen, S. A., et al., J. Clin. Microbiol. 14:441-445, 1981) tests using whole T. pallidum antigen extracts and a variety of T. pallidum recombinant protein tests (Gerber, A., et al., Immunobiology 196:535-549, 1996; Hagedorn, H. J., et al., J. Clin. Microbiol. 40:973-978, 2002; Ijsselmuiden, O. E., et al., Eur. J. Clin. Microbiol. Infect. Dis. 8:716-721, 1989; Ijsselmuiden, O. E., et al., J. Clin. Microbiol. 27:152-157, 1989; Larsen, S. A., et al., J. Clin. Microbiol. 14:441-445, 1981; Peterson, K. M., et al., J. Exp. Med. 164:1160-1170, 1986; Radolf, J. D., et al., J. Infect. Dis. 153:1023-1027, 1986; Rodriguez, I., et al., Mem. Inst. Oswaldo Cruz 97:347-349, 2002; Sambri, V., et al., Clin. Microbiol. Infect. 7:200-205, 2001; Sato, N. S., et al., Rev. Inst. Med. Trop. Sao Paulo 41:115-118, 1999; Schmidt, B. L., et al., J. Clin. Microbiol. 38:1279-1282, 2000; Schouls, L. M., et al., Infect. Immun. 57:2612-2623, 1989; Young, H., Dermatol. Clin. 16:691-698, 1998; Young, H. et al., Int. J. STD AIDS 11:288-291, 2000; Young, H, et al., Int. J. STD AIDS 9:196-200, 1998; Young, H., et al., J. Clin. Microbiol. 36:913-917; Zrein, M., et al., J. Clin. Microbiol. 33:525-527).

Improved syphilis diagnostic tests are, nonetheless, required because the RPR and VDRL tests give false positives, require a secondary specific test, and are not sensitive in detecting early syphilis. Moreover, many if not all of the available recombinant Treponema proteins that could, in principle, be used in a syphilis diagnostic test do not react with antibodies from syphilitic individuals with sufficient specificity.

### SUMMARY OF THE INVENTION

In accordance with the foregoing, the present inventors have identified T. pallidum proteins and fragments thereof that can be used, alone or in combination, in syphilis diagnostic tests. The T. pallidum proteins are of three types: Tp92 proteins, Tp0453 proteins and Gpd proteins. Each of the foregoing three types of proteins is believed to be located, in vivo, in the outer membrane of T. pallidum, which may explain their immunogenicity. SEQ ID NO:1 sets forth the nucleic acid sequence of a gene encoding the representative Tp92 protein having the amino acid sequence set forth in SEQ ID NO:2. SEQ ID NO:3 sets forth the nucleic acid sequence of a gene encoding the representative Tp0453 protein having the amino acid sequence set forth in SEQ ID NO:4. SEQ ID NO:5 sets forth the nucleic acid sequence of a gene encoding the representative Gpd protein having the amino acid sequence set forth in SEQ ID NO:6.

Thus, in one aspect, the present invention provides methods for specific detection of antibodies resulting from exposure to or infection with Treponema pallidum in a biological sample from a test subject. The methods of this aspect of the invention each include the steps of (a) contacting the biological sample with one or more isolated polypeptides having a sequence at least 95% identical to a polypeptide selected from the group consisting of (i) a polypeptide fragment of a Tp92 protein having no more than 740 amino acids, the fragment having the sequence of SEQ ID NO:12 or an immunogenic fragment thereof, (ii) a polypeptide fragment of a Tp0453 protein having no more than 140 amino acids, the fragment having the sequence of SEQ ID NO:23 or an immunogenic fragment thereof, and (iii) a polypeptide fragment of a Gpd protein having no more than 195 amino acids, the fragment having the sequence of SEQ ID NO:28 or an immunogenic fragment thereof; and (b) detecting whether antigen-antibody binding has occurred between the polypeptide and an antibody component of the biological sample in which the detection of antigen-antibody binding indicates the presence of antibodies in the biological sample and is indicative of exposure to or infection of the test subject with Treponema pallidum.

In another aspect, the present invention provides a method for specific detection of antibodies resulting from exposure to or infection with Treponema pallidum in a biological sample from a test subject, the method comprising the steps of (a) contacting the biological sample with one or more isolated polypeptides having no more than 200 amino acids and a sequence at least 95% identical to a polypeptide selected from the group consisting of (i) a polypeptide fragment of a Tp92 protein having the sequence of SEQ ID NO:13 or an immunogenic fragment thereof, (ii) a polypeptide fragment of a Tp0453 protein having the sequence of SEQ ID NO:23 or an immunogenic fragment thereof, and (iii) a polypeptide fragment of a Gpd protein having the sequence of SEQ ID NO:28 or an immunogenic fragment thereof; and (b) detecting whether antigen-antibody binding has occurred between the polypeptide and an antibody component of the biological sample in which the detection of antigen-antibody binding indicates the presence of antibodies in the biological sample and is indicative of exposure to or infection of the test subject with Treponema pallidum.

In another aspect, the present invention provides kits that each include (a) one or more isolated polypeptides having a sequence at least 95% identical to a polypeptide selected from the group consisting of (i) a polypeptide fragment of a Tp92 protein having no more than 740 amino acids, the fragment having the sequence of SEQ ID NO:12 or an immunogenic fragment thereof, (ii) a polypeptide fragment of a Tp0453 protein having no more than 140 amino acids, the fragment having the sequence of SEQ ID NO:23 or an immunogenic fragment thereof, and (iii) a polypeptide fragment of a Gpd protein having no more than 195 amino acids, the fragment having the sequence of SEQ ID NO:28 or an immunogenic fragment thereof; (b) reagents for labeling antibody bound to the isolated polypeptide; and (c) written indicia providing a user with instructions for use of the kit to determine whether a human subject is infected with T. pallidum.

In another aspect, the present invention provides isolated polypeptides derived from the Tp92, Tp0452, and Gpd proteins that bind antibodies from sera of human subjects infected with T. pallidum. Thus, in one aspect, the invention provides isolated polypeptide fragments having the amino acid sequences set forth in SEQ ID NO:7, SEQ ID NO:12, SEQ ID NO:13, and SEQ ID NO:21, which are fragments of the Tp92 protein having the amino acid sequence set forth in SEQ ID NO:2. In a related aspect, the present invention provides isolated polypeptides having the amino acid sequences set forth in SEQ ID NOs:23, 26 and 27, which are fragments of the Tp0453 protein having the amino acid sequence set forth in SEQ ID NO:4. In a further related aspect, the present invention provides isolated polypeptides having the amino acid sequences set forth in SEQ ID NOs:28 and 30, which are fragments of the Gpd protein having the amino acid sequence set forth in SEQ ID NO:6. The isolated polypeptides of the invention can be used, for example, in the methods and kits of the invention.

In another aspect, the invention provides isolated polypeptides comprising a sequence at least 95% identical to a polypeptide selected from the group consisting of (i) a polypeptide fragment of a Tp92 protein having no more than 740 amino acids, the fragment having the sequence of SEQ ID NO:12 or an immunogenic fragment thereof; (ii) a polypeptide fragment of a Tp0453 protein having no more than 140 amino acids, the fragment having the sequence of SEQ ID NO:23 or an immunogenic fragment thereof; and (iii) a polypeptide fragment of a Gpd protein having no more than 195 amino acids, the fragment having the sequence of SEQ ID NO:28 or an immunogenic fragment thereof.

In another aspect, the invention provides expression vectors comprising a nucleic acid sequence that encodes a polypeptide of the invention. Thus, in one aspect, the present invention provides an expression vector comprising a nucleic acid sequence that encodes a polypeptide comprising a sequence at least 95% identical to a polypeptide selected from the group consisting of (i) a polypeptide fragment of a Tp92 protein having no more than 740 amino acids, the fragment having the sequence of SEQ ID NO:12 or an immunogenic fragment thereof; (ii) a polypeptide fragment of a Tp0453 protein having no more than 140 amino acids, the fragment having the sequence of SEQ ID NO:23 or an immunogenic fragment thereof; and (iii) a polypeptide fragment of a Gpd protein having no more than 195 amino acids, the fragment having the sequence of SEQ ID NO:28 or an immunogenic fragment thereof. The expression vectors of the invention can be used, for example, to transform or transfect cells that produce the polypeptides used in the methods and kits of the invention.

In another aspect, the present invention provides livings cells such as prokaryotic or eukaryotic cells that comprise an expression vector of the invention. The living cells comprising an expression vector of the invention can be used to produce the polypeptides used in the methods and kits of the invention.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a bar chart showing the reactivity of a panel of sera from human syphilis patients against Tp92 (SEQ ID NO:2) and 15 fragments of Tp92 (SEQ ID NOs:7-21). Shaded bars represent the reactivity of human serum from syphilis patients. Unshaded bars represent the reactivity of human serum from subjects who were not infected with T. pallidum;

FIG. 2 shows a bar chart showing the reactivity of a panel of sera from rabbits (artificially infected with T. pallidum) against Tp92 (SEQ ID NO:2) and 15 fragments of Tp92 (SEQ ID NOs:7-21). Shaded bars represent the reactivity of serum from rabbits infected with T. pallidum. Unshaded bars represent the reactivity of serum from rabbits that were not infected with T. pallidum;

FIG. 3A shows a schematic diagram of the full-length Tp0453 protein (SEQ ID NO:4) and fragments Tp0453.1-Tp0453.6 (SEQ ID NOs:22-27), as described in Example 2;

FIG. 3B shows a bar chart showing the reactivity of a panel of pooled sera from human syphilis patients against Tp0453 (SEQ ID NO:4) and fragments Tp0453.1-Tp0453.6 (SEQ ID NOs:22-27), as described in Example 2;

FIG. 4 shows a bar chart showing the reactivity of a panel of pooled sera with fragment Tp0453.2 (SEQ ID NO:23) after pre-absorption with fragments Tp0453.2 (SEQ ID NO:23), Tp0453.5 (SEQ ID NO:26), and Tp0453.6 (SEQ ID NO:27), as described in Example 2;

FIG. 5A shows a schematic diagram of the full-length Gpd protein (SEQ ID NO:6) and fragments Gpd.1-Gpd.6 (SEQ ID NOs:28-33), as described in Example 3;

FIG. 5B shows a bar chart showing the reactivity of a panel of pooled sera from human syphilis patients against Gpd (SEQ ID NO:6) and fragments Gpd.1-Gpd.6 (SEQ ID NOs:28-33), as described in Example 3;

FIG. 6 shows titration curves for the Gpd fragments described in FIG. 3A, where the x-axis represents 1:2 serial dilutions from 1:100 to 1:3200 and IHS refers to the infected human serum patient pool as described in Example 3;

FIG. 7 shows graph plotting mean absorbance values from individual serum samples of human subjects at various stages of syphilis infection obtained by ELISA using wells coated with a mixture of peptide fragments Tp92.F7 (SEQ ID NO:13), Tp0453.2 (SEQ ID NO:23), and Gpd.1 (SEQ ID NO:28), where the number (n) of individuals for each group is shown above each stage, the boxes represent the second and third quartile values, whiskers represent one standard deviation from the mean, values outside the first standard deviation are shown individually, and the dashed line represents three times the standard deviation of the mean of uninfected control (normal) subjects, as described in Example 4; and

FIG. 8 shows graph plotting mean absorbance values from individual serum samples of human subjects infected with various spirochete bacteria and normal controls obtained by ELISA using wells coated with a mixture of peptide fragments Tp92.F7 (SEQ ID NO:13), Tp0453.2 (SEQ ID NO:23), and Gpd.1 (SEQ ID NO:28), as described in Example 5. The symbols are as described in FIG. 7 and abbreviations on the X-axis are as follows: Lepto=Leptospirosis, LD=Lyme Disease, RF=Relapsing Fever, Perio=Periodontal Disease, and Normal=uninfected control subjects.

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art.

As used herein, the term “Tp92 protein” refers to a protein that is at least 70% identical to the Tp92 protein having the amino acid sequence set forth in SEQ ID NO:2 (having GenBank accession number AF152012).

As used herein, the term “Tp0453 protein” refers to a protein that is at least 70% identical to the Tp0453 protein having the amino acid sequence set forth in SEQ ID NO:4 (having GenBank accession number NC_{—}000919).

As used herein, the term “Gpd protein” refers to glycerophosphodiester phosphodiesterase, a protein that catalyzes the hydrolysis of glycerophosphodiesters from phospholipids and triglycerides to glycerol 3-phosphate and that is at least 70% identical to the Gpd protein having the amino acid sequence set forth in SEQ ID NO:6 (having GenBank accession number AF127421).

As used herein, the term “T. pallidum” refers to Treponema pallidum, subspecies pallidum, the spirochete bacterium that causes syphilis.

As used herein, the term “isolated polypeptide” refers to a polypeptide that is substantially (such as at least 99% pure) or completely free from components that normally accompany it as found in its native state. Purity and homogeneity may be determined using analytical chemistry techniques such as polyacrylamide gel electrophoresis or high performance liquid chromatography. An isolated polypeptide is typically the predominant or sole band when visualized using polyacrylamide gel electrophoresis.

As used herein, the terms “polypeptide fragment,” “peptide fragment,” “recombinant fragment,” or grammatical equivalents thereof refer to an isolated portion of a full-length Tp92, Tp0453, or Gpd protein and also include polypeptides that are at least 70%, at least 80%, at least 90%, at least 95%, and at least 99% identical to a polypeptide fragment disclosed herein.

As used herein, the term “full-length protein” and grammatical equivalents thereof includes both the full-length protein encoded by an mRNA or cDNA and a protein in which the amino-terminal signal sequence has been removed during isolation and expression. Thus, as used herein, the term “polypeptide fragment” does not include the full-length protein or the full-length protein minus the signal sequence. For example, a fragment of a Tp92 protein may consist of no more than about 740 amino acids, no more than about 567 amino acids, no more than about 378 amino acids, and no more than about 195 amino acids. By way of further example, the fragment of a Tp92 protein may include amino acids 26-764 (SEQ ID NO:12), amino acids 26-592 (SEQ ID NO:7) amino acids 26-403 (SEQ ID NO:21) or amino acids 26-220 (SEQ ID NO:13) of the full-length Tp92 protein or immunogenic fragments thereof. As used herein, the term “immunogenic fragment” refers to a polypeptide fragment or sub-fragment of a polypeptide that specifically binds antibodies present in a biological sample from a test subject.

A fragment of a Tp0453 protein may consist of no more than about 140 amino acids, no more than about 81 amino acids, and no more than about 79 amino acids. For example, the fragment of a Tp0453 protein may include amino acids 149-287 (SEQ ID NO:23) amino acids 149-228 (SEQ ID NO:26) or amino acids 209-287 (SEQ ID NO:27) of the full-length Tp0453 protein or immunogenic fragments thereof.

A fragment of a GPD protein may consist of no more than about 191 amino acids and no more than about 103 amino acids. For example, the fragment of a GPD protein may include amino acids 1-190 (SEQ ID NO:28) or amino acids 1-102 (SEQ ID NO:30) of the full-length Gpd protein or immunogenic fragments thereof.

The terms “percent identical” or “percent identity,” or grammatical equivalents thereof, as applied to a polypeptide are the percentage of amino acid residues in a candidate polypeptide sequence that is identical with a subject polypeptide sequence after aligning the sequences to achieve the maximum percent identity. No gaps are introduced into the candidate polypeptide sequence in order to achieve the best alignment. Amino acid sequence identity can be determined, for example, in the following manner. The subject polypeptide sequence is used to search a polypeptide sequence database, such as the GenBank database (maintained by the National Center for Biotechnology Information, National Library of Medicine, Building 38A, Bethesda, Md. 20894, U.S.A.) using the BLASTP program. The program is used in the ungapped mode. Default filtering is used to remove sequence homologies due to regions of low complexity. The default parameters of BLASTP are utilized. Filtering for sequences of low complexity utilize the SEG program. The BLASTP program identifies polypeptide sequences in the database that have a defined level of identity to the subject polypeptide sequence.

As used herein, the term “bodily fluid” refers to a liquid that is a component of the body of a test subject. Examples of bodily fluids include whole blood, blood serum, blood plasma, and saliva. However, other suitable bodily fluids may be used.

As used herein, the term “test subject” refers to a human who may be or is suspected of being infected with T. pallidum. The term also includes humans who have been diagnosed with symptoms of syphilis by other methods and may require confirmatory diagnostic tests using the methods provided herein. The term further includes humans who may not be infected or have not been diagnosed with syphilis but who are being tested for T. pallidum infection—for example, to confirm the absence of infection by T. pallidum.

As used herein, the term “sensitivity” as applied to an assay or test employing polypeptides disclosed herein means the ability of the test or assay to detect true positives—for example, to detect human subjects who were infected with T. pallidum. Sensitivity can be calculated by the formula sensitivity=number of true positives/number of true positives+number of false negatives.

As used herein, the term “specificity” as applied to an assay or test employing polypeptides disclosed herein means the ability of the test or assay to detect true negatives—for example, to exclude human subjects who are not infected with T. pallidum. Specificity can be calculated by the formula specificity=number of true negatives/number of true negatives+number of false positives.

As used herein, the term “positive predictive value” refers to the ability of the assay or test to predict human subjects who are infected with T. pallidum. Positive predictive value can be calculated by the formula positive predictive value=number of true positives/number of true positives+number of false positives.

As used herein, the term “negative predictive value” refers to the ability of the assay or test to exclude subjects who are not infected with T. pallidum. Negative predictive value can be calculated by the formula negative predictive value=number of true negatives/number of true negatives+number of false negatives.

The above parameters concerning sensitivity, selectivity, positive predictive value, and negative predictive value are usually compared to a “gold standard” test, such as the FTA-Ab, MHA-TP, and TPHA tests.

In one aspect, the present invention provides methods for specific detection of antibodies resulting from exposure to or infection with Treponema pallidum in a biological sample from a test subject. The methods of this aspect of the invention each include the steps of (a) contacting the biological sample with one or more isolated polypeptides having a sequence at least 95% identical to a polypeptide selected from the group consisting of (i) a polypeptide fragment of a Tp92 protein having no more than 740 amino acids, the fragment having the sequence of SEQ ID NO:12 or an immunogenic fragment thereof; (ii) a polypeptide fragment of a Tp0453 protein having no more than 140 amino acids, the fragment having the sequence of SEQ ID NO:23 or an immunogenic fragment thereof; and (iii) a polypeptide fragment of a Gpd protein having no more than 195 amino acids, the fragment having the sequence of SEQ ID NO:28 or an immunogenic fragment thereof; and (b) detecting whether antigen-antibody binding has occurred between the polypeptide and an antibody component of the biological sample in which the detection of antigen-antibody binding indicates the presence of antibodies in the biological sample and is indicative of exposure to or infection of the test subject with Treponema pallidum.

Regarding the above aspect, in one embodiment the methods include contacting a biological sample from a test subject with an isolated polypeptide having a sequence at least 95% identical to a polypeptide fragment of a Tp92 protein having no more than 740 amino acids, wherein the fragment comprises SEQ ID NO:12 or an immunogenic fragment thereof. Representative examples of immunogenic fragments of SEQ ID NO:12 include SEQ ID NO:7, SEQ ID NO:13, and SEQ NO:21. However, other suitable immunogenic fragments of SEQ ID NO:12 may be used. In another embodiment, the methods include contacting a biological sample from a test subject with an isolated polypeptide having a sequence at least 95% identical to a polypeptide fragment of a Tp04523 protein having no more than 140 amino acids, wherein the fragment comprises SEQ ID NO:23 or an immunogenic fragment thereof. Representative examples of immunogenic fragments of SEQ ID NO:23 include SEQ ID NO:26 and SEQ ID NO:27. However, other suitable immunogenic fragments of SEQ ID NO:23 may be used. In another embodiment, the methods include contacting a biological sample from a test subject with an isolated polypeptide having a sequence at least 95% identical to a polypeptide fragment of a Gpd protein having no more than 195 amino acids, wherein the fragment comprises SEQ ID NO:28 or an immunogenic fragment thereof. Representative examples of immunogenic fragments of SEQ ID NO:28 include SEQ ID NO:30. However, other suitable immunogenic fragments of SEQ ID NO:28 may be used. In some embodiments, the methods include contacting a biological sample from a test subject with two or more of the above immunogenic polypeptide fragments. In other embodiments, the methods include contacting a biological sample from a test subject with an isolated polypeptide having a sequence at least 95% identical to an immunogenic polypeptide fragment from a Tp92 protein, an immunogenic polypeptide fragment from a Tp0453 protein, and an immunogenic polypeptide fragment from a Gpd protein.

In another aspect, the present invention provides a method for specific detection of antibodies resulting from exposure to or infection with Treponema pallidum in a biological sample from a test subject, the method comprising the steps of (a) contacting the biological sample with one or more isolated polypeptides having no more than 200 amino acids and a sequence at least 95% identical to a polypeptide selected from the group consisting of (i) a polypeptide fragment of a Tp92 protein having the sequence of SEQ ID NO:13 or an immunogenic fragment thereof; (ii) a polypeptide fragment of a Tp0453 protein having the sequence of SEQ ID NO:23 or an immunogenic fragment thereof; and (iii) a polypeptide fragment of a Gpd protein having the sequence of SEQ ID NO:28 or an immunogenic fragment thereof; and (b) detecting whether antigen-antibody binding has occurred between the polypeptide and an antibody component of the biological sample in which the detection of antigen-antibody binding indicates the presence of antibodies in the biological sample and is indicative of exposure to or infection of the test subject with Treponema pallidum.

In the practice of the methods of the present invention, a fragment of a Tp92 protein and/or a fragment of a Tp0453 protein and/or a fragment of a Gpd protein is/are contacted with a biological sample from a test subject. Typically, each fragment is attached to a substrate and the substrate is contacted with a liquid composition (such as whole blood, plasma, serum, saliva or another bodily fluid) that may contain the antibodies, for a period of time sufficient to permit the antibodies to bind to the fragment(s). For example, the substrate can be completely or partially immersed in the liquid composition. Binding of the antibodies to the immobilized protein fragment(s) indicates that the test subject who produced the antibodies is infected with T. pallidum. Further, because the sera of test subjects who were previously treated for syphilis may still contain antibodies produced in response to infection with T. pallidum, binding of the antibodies to the immobilized protein fragment(s) indicates that the test subject who produced the antibodies was previously infected with or exposed to T. pallidum.

One method of detecting the presence of antibodies bound to the polypeptide(s) is an immunoassay. One having ordinary skill in the art can readily appreciate the multitude of ways to practice an immunoassay to detect the presence of antibodies bound to the polypeptide(s). Various immunoassay procedures are described in J. Goers, “Immunochemical Techniques Laboratory Manual,” Academic Press (1993), and “Current Protocols in Immunology,” Coligan, J. E., et al., eds., John Wiley and Sons Publishers, 2005 (Vols. 1-5), New York, which publications are incorporated herein by reference.

For example, an indirect antibody capture immunoassay (a form of enzyme-linked immunosorbent assay (ELISA)) can be used to detect the presence of antibody bound to the isolated polypeptide(s). An example of an indirect antibody capture immunoassay is described in Chapter 10 of J. Goers, “Immunochemical Techniques Laboratory Manual,” supra. For example, the wells of a plastic microtiter plate are coated with an isolated fragment of a Tp92 protein, a Tp0453 protein, or a Gpd protein and serum from a human being is added to one or more of the coated wells. For example, serum from up to 96 different human beings (suspected of suffering from syphilis) can be added to the coated wells of a 96-well plastic microtiter plate. If the serum contains antibody that binds to one of the isolated fragments of a Tp92 protein, a Tp0453 protein, or a Gpd protein, then the antibody binds to the fragment(s) in the well and is thereafter detected by a detectably labeled molecule that selectively binds to antibodies, such as labeled protein A, or a labeled anti-class-specific antibody, or labeled anti-subclass specific antibody. A class-specific antibody specifically binds to a particular class of antibodies (e.g., IgM antibody, IgG antibody, or IgA antibody). A subclass-specific antibody specifically binds to a particular subclass of antibodies (e.g., subclasses IgG1, IgG2a, IgG2b, IgG3, or IgG4).

Examples of substrates to which the polypeptide(s) may be bound include nitrocellulose, glass, polystyrene, polypropylene, polyethylene, dextran, nylon, amylases, natural and modified celluloses, polyacrylamides, agaroses, and magnetite. The substrate configuration may be, for example, spherical (e.g., a bead), cylindrical (e.g., the inside surface of a test tube or the external surface of a rod), or flat, such as a sheet or test strip (e.g., plastic test strip). Again by way of example, polypeptide(s) may be bound to the wells of a microtiter plate. Polypeptide(s) may be covalently or non-covalently bound to a substrate.

Examples of enzymes that can be used to detectably label molecules that selectively bind to antibodies include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase, and acetylcholinesterase.

Examples of radioactive isotopes that can be used to detectably label molecules that selectively bind to antibodies include, but are not limited to, ³H, ¹²⁵I, ¹³¹I, ³⁵S, and ¹⁴C. The radioactive isotope can be detected by such means as the use of a gamma counter or a scintillation counter or by autoradiography.

Fluorescent compounds can also be used to detectably label molecules that selectively bind to antibodies. Representative examples of useful fluorescent compounds include fluorescein isothiocyanate, rhodamine, phycoerythrin, phycocyanin, allophycocyanin, o-phthaldehyde, and fluorescamine. When the fluorescent-labeled molecule is exposed to light of the proper wave length, its presence can be detected due to its fluorescence.

Again by way of example, fluorescence-emitting metals such as ¹⁵²Eu or others of the lanthanide series, can also be used to detectably label molecules that selectively bind to antibodies. These metals can be attached using such metal chelating groups as diethylenetriaminepentaacetic acid (DTPA) or ethylenediamine-tetraacetic acid (EDTA).

Chemiluminescent compounds (e.g., luminol, isoluminol, theromatic acridinium ester, imidazole, acridinium salt, and oxalate ester) and bioluminescent compounds (e.g., luciferin, luciferase, and aequorin) can also be used to detectably label molecules that selectively bind to antibodies.

Molecules that selectively bind to antibodies (e.g., protein A) can also be linked to biotin and the biotin can be detected by avidin or streptavidin that is detectably labeled.

As understood by those of ordinary skill in the art, positive and negative controls are typically performed in which known amounts of polypeptide and no polypeptide, respectively, are used in assays being performed in parallel with the test assay. Thus, for example, if a sample of human serum provided a positive result in a negative control, then that result would be considered artifactual.

The isolated polypeptides used in the practice of the invention may be selected from the following group: (1) a fragment corresponding to amino acids 26-220 (SEQ ID NO:13) of the full-length Tp92 protein; (2) a fragment corresponding to amino acids 26-403 (SEQ ID NO:21) of the full-length Tp92 protein; (3) a fragment corresponding to amino acids 26-592 (SEQ ID NO:7) of the full-length Tp92 protein; (4) a fragment corresponding to amino acids 26-764 (SEQ ID NO:12) of the full-length Tp92 protein; (5) a fragment corresponding to amino acids 149-287 (SEQ ID NO:23) of the full-length Tp0453 protein; (6) a fragment corresponding to amino acids 149-228 (SEQ ID NO:26) of the full-length Tp0453 protein; (7) a fragment corresponding to amino acids 209-287 (SEQ ID NO:27) of the full-length Tp0453 protein; (8) a fragment corresponding to amino acids 1-190 (SEQ ID NO:28) of the full-length Gpd protein; and (9) a fragment corresponding to amino acids 1-102 (SEQ ID NO:30) of the full-length Gpd protein

One type of isolated polypeptide or more than one type of isolated polypeptide may be used in the practice of the invention. For example, the combination of a fragment of Tp92 corresponding to SEQ ID NO:13, a fragment of Tp0453 corresponding to SEQ ID NO:23, and a fragment of Gpd corresponding to SEQ ID NO:28 may be used in the practice of the invention. Again by way of example, the combination of two members of the following group may be used in the practice of the invention—a fragment of Tp92 corresponding to SEQ ID NO:13, a fragment of Tp0453 corresponding to SEQ ID NO:23, and a fragment of Gpd corresponding to SEQ ID NO:28. In other embodiments, an isolated polypeptide fragment described above may be used in combination with a full-length Tp92, a full-length Tp0453, and/or a full length Gpd protein in the practice of the invention.

Again by way of example, a hybrid protein that includes a polypeptide fragment of a Tp92 protein, a Tp0453 protein, and a Gpd protein may be used. Hybrid proteins may be made, for example, using standard nucleic acid cloning and manipulation techniques, such as the techniques described in Ausubel et al., supra. For example, to make a hybrid protein that includes a defined portion of a Tp92 protein, a defined portion of a Tp0453 protein, and a defined portion of a Gpd protein, the coding sequence for each of the foregoing portions can be excised (using restriction enzymes) from a Tp92 gene (e.g., the gene having the nucleic acid sequence set forth in SEQ ID NO:1), a Tp0453 gene (e.g., the gene having the nucleic acid sequence set forth in SEQ ID NO:3), and a Gpd gene (e.g., the gene having the nucleic acid sequence set forth in SEQ ID NO:5), respectively, and the excised portions are ligated together to form a hybrid nucleic acid molecule that is then ligated into an expression vector. The resulting, recombinant, expression vector is introduced into a suitable host cell (e.g., yeast cells) and the encoded, hybrid, protein is expressed therein and purified from the cells. Again by way of example, the required gene portions can be amplified using PCR and then ligated together and ligated into an expression vector.

Useful expression vectors can include transcriptional and translational regulatory sequences. In general, the transcriptional and translational regulatory sequences may include, but are not limited to, promoter sequences, ribosomal binding sites, transcriptional start and stop sequences, translational start and stop sequences, and enhancer or activator sequences. Promoter sequences may be, for example, constitutive or inducible promoters. The promoters may be, for example, naturally-occurring promoters or hybrid promoters. In addition, an expression vector typically contains a selectable marker gene to allow the selection of transformed host cells. Vectors useful for expressing a desired polypeptide can be any type of vector, including plasmid vectors and viral vectors.

It is also possible to use protein synthesis techniques to synthesize a protein that includes portions of one, two, or all three of a Tp92 protein, a Tp0453 protein, and a Gpd protein. Protein synthesis techniques are described, for example, in Aimoto, S., “Contemporary Methods for Peptide and Protein Synthesis,” Current Organic Chemistry 5:45-87 (2001).

In another aspect, the present invention provides kits that each include (a) one or more isolated polypeptides having a sequence at least 95% identical to a polypeptide selected from the group consisting of (i) a polypeptide fragment of a Tp92 protein having no more than 740 amino acids, the fragment having the sequence of SEQ ID NO:12 or an immunogenic fragment thereof; (ii) a polypeptide fragment of a Tp0453 protein having no more than 140 amino acids, the fragment having the sequence of SEQ ID NO:23 or an immunogenic fragment thereof; and (iii) a polypeptide fragment of a Gpd protein having no more than 195 amino acids, the fragment having the sequence of SEQ ID NO:28 or an immunogenic fragment thereof; (b) reagents for labeling antibody bound to the isolated polypeptide; and (c) written indicia providing a user with instructions for use of the kit to determine whether a human subject is infected with T. pallidum. The kits may optionally include a liquid for washing the substrate after the substrate has been contacted with a bodily fluid from a human being and before the strip is contacted with the reagents for labeling antibody bound to the isolated polypeptide. The kits may also include packaging that contains the foregoing components.

The isolated polypeptide(s) may be covalently or non-covalently bound to the substrate. Representative examples of useful substrates are described in connection with the methods of the invention for determining whether a human subject is infected with T. pallidum. Representative reagents for labeling antibodies are also described in connection with the methods of the invention for determining whether a human subject is infected with T. pallidum. The kit also includes written indicia providing a user with instructions for use of the kit to determine whether a human subject is infected with T. pallidum. Thus, for example, the written indicia might be located upon a paper insert present within packaging that is included as part of the kit or may be located upon part of the packaging. The written indicia may provide representative values for the amount of antibody that is bound to the isolated polypeptide(s) that indicates that the human subject is infected with T. pallidum. Additionally, the kit may optionally include depictions or photographs that represent the appearance of positive and negative results (such as a color change visible on the substrate that indicates the presence of antibodies bound to the polypeptides on the substrate).

Typically, the kits of the present invention include controls, such as a substrate to which no polypeptide is bound, and a substrate that bears a polypeptide that is selected so that it is not recognized by antibodies directed against T. pallidum.

In another aspect, the present invention provides isolated polypeptides comprising portions or fragments of the Tp92, Tp0452, and Gpd proteins that bind antibodies from sera of human subjects infected with T. pallidum. The isolated polypeptides can be used, for example, in the methods and kits of the invention. In one embodiment, the present invention provides the isolated polypeptides having the amino acid sequences set forth in SEQ ID NOs:7-21, which are fragments of the Tp92 polypeptide having the amino acid sequence set forth in SEQ ID NO:2. In particular, as described more fully in Example 1 herein, the isolated polypeptide having the amino acid sequence set forth in SEQ ID NO:13 is a highly immunogenic fragment of Tp92 (SEQ ID NO:2).

In another embodiment, the present invention provides isolated polypeptides having the amino acid sequences set forth in SEQ ID NOs:23 and 26, which are fragments of the Tp0453 protein having the amino acid sequence set forth in SEQ ID NO:4. As described more fully in Example 2 herein, the isolated polypeptides having the amino acid sequences set forth in SEQ ID NOs:23, 26, and 27 bind antibodies present in sera from human subjects infected with T. pallidum.

In a further embodiment, the present invention provides isolated polypeptides having the amino acid sequences set forth in SEQ ID NOs:28 and 30, which are fragments of the Gpd protein having the amino acid sequence set forth in SEQ ID NO:6. As described more fully in Example 3 herein, the isolated polypeptides having the amino acid sequences set forth in SEQ ID NOs:28 and 30 bind antibodies present in sera from human subjects infected with T. pallidum.

In another aspect, the invention provides combinations and/or mixtures of isolated polypeptide fragments comprising portions of the Tp92, Tp0452, and Gpd proteins that bind antibodies from sera of human subjects infected with T. pallidum. As described more fully in Examples 4 and 5 herein, combinations and mixtures of isolated polypeptide fragments comprising portions of the Tp92, Tp0452, and Gpd proteins show high sensitivity and specificity in the serodiagnosis of various stages of syphilis in human subjects. In one embodiment, the combinations and/or mixtures of isolated polypeptides are selected from polypeptides having a sequence at least 95% identical to a polypeptide selected from the group consisting of SEQ ID NO:13, SEQ ID NO:23, SEQ ID NO:26, SEQ ID NO:27, SEQ ID NO:28, and SEQ ID NO:30. In another embodiment, the combinations and/or mixtures of isolated polypeptides are selected from polypeptides having a sequence at least 95% identical to a polypeptide selected from the group consisting of SEQ ID NO:13, SEQ ID NO:23, and SEQ ID NO:28.

In another aspect, the invention provides combinations and/or mixtures of isolated polypeptides comprising a sequence at least 95% identical to a polypeptide selected from the group consisting of (i) a polypeptide fragment of a Tp92 protein having no more than 740 amino acids, the fragment having the sequence of SEQ ID NO:12 or an immunogenic fragment thereof; (ii) a polypeptide fragment of a Tp0453 protein having no more than 140 amino acids, the fragment having the sequence of SEQ ID NO:23 or an immunogenic fragment thereof; and (iii) a polypeptide fragment of a Gpd protein having no more than 195 amino acids, the fragment having the sequence of SEQ ID NO:28 or an immunogenic fragment thereof.

The polypeptide fragments of the present invention provide advantages over the use of full-length proteins. For example, polypeptide fragments can typically be made in greater quantities than full-length proteins, which results in increased purity of the polypeptide fragments. Further, the polypeptide fragments have fewer potential cross-reactive epitopes than full-length proteins, resulting in increased specificity. Moreover, one of skill in the art would recognize that expression of full-length proteins is often toxic to bacterial cells and that expression of polypeptide fragments of T. pallidum proteins may overcome the toxicity (Van Voorhis, W. C., et al., J. Clin. Microbiol. 41:3668-3674, 2003). The following examples merely illustrate the best mode now contemplated for practicing the invention, but should not be construed to limit the invention.

### Example 1

This example describes the expression of the 15 Tp92 polypeptide fragments having the amino acid sequences set forth in SEQ ID NOs:7-21 in E. coli and the purification of the 15 Tp92 polypeptide fragments (SEQ ID NOs:7-21) from E. coli. This example also describes the results of experiments to identify which of the 15 Tp92 polypeptide fragments (SEQ ID NOs:7-21) are bound by antibodies from human syphilis patients.

Fifteen recombinant protein fragments (SEQ ID NOs:7-21) were created that span the entire length of the mature Tp92 protein (SEQ ID NO:2). Table 1 shows the boundaries of these fragments (numbering is from the first amino acid at the N-terminus of the mature Tp92 protein (SEQ ID NO:2)).

TABLE 1

| | Number of First and | |
|---|---|---|
| Fragment | Last Amino Acid | |
| Number | Residue | SEQ ID NO: |
| F1 | 26-592 | 7 |
| F2 | 593-837 | 8 |
| F3 | 593-759 | 9 |
| F4 | 749-837 | 10 |
| F5 | 785-837 | 11 |
| F6 | 26-764 | 12 |
| F7 | 26-220 | 13 |
| F8 | 221-592 | 14 |
| F9 | 26-123 | 15 |
| F10 | 124-220 | 16 |
| F11 | 221-344 | 17 |
| F12 | 345-456 | 18 |
| F13 | 457-592 | 19 |
| F14 | 404-775 | 20 |
| F15 | 26-403 | 21 |

The 15 Tp92 peptides (SEQ ID NOs:7-21) were prepared by subcloning PCR amplified portions of the Tp92 gene into an expression vector containing 6-histidines at the N-terminus. The recombinant vectors were transformed into E. coli, and the encoded Tp92 peptides (SEQ ID NOs:7-21) were expressed in the E. coli that was then lysed. The expressed Tp92 peptides (SEQ ID NOs:7-21) were purified from the E. coli lysates using Nickel-affinity chromatography.

Using an ELISA-based immunological detection system, a panel of serum collected from diagnosed syphilis patients was tested against these recombinant protein fragments (SEQ ID NOs:7-21) to determine where the immunological reactivity is focused within the molecule. The ELISA was carried out by coating 96 well plates (Maxisorp F9; Costar) overnight at 4° C. with 50 μl of the recombinant T. pallidum proteins per well in phosphate-buffered saline (PBS), pH 7.4, with 0.1% sodium dodecyl sulfate at concentrations of 2 μg/ml of recombinant proteins and peptides. Plates were blocked at room temperature for 2 hours with 4% milk in phosphate buffered saline (PBS). Human sera or rabbit sera was diluted 1:200 (Gpd assays) or 1:100 (all other assays) in dilution buffer (4% milk and 0.2% Triton X-100 in PBS). The diluted sera were adsorbed overnight at 4° C. with a 0.5% (vol/vol) lysate of E. coli expressing an irrelevant Trypanosoma cruzi recombinant protein (SA85-1.1) in pRSET (Kahn, S. J., and M. Wleklinski., J. Immunol. 159:4444-4451 (1997)). This adsorption step was omitted from the sera tested for reactivity to Gpd since preliminary experiments with Gpd showed that this step had no effect on background reactivity. Samples were spun at 4° C. at 12,000×g for 10 minutes and 50 μl of each serum was added to triplicate wells and incubated for 1 hour at room temperature. After washing, 50 μl of a 1:3,000 dilution of goat anti-human (gamma specific) F(ab′)₂ peroxidase (Sigma-Aldrich, St. Louis, Mo.) was applied and incubated at room temperature for 1 hour. Plates were developed for 30 minutes at room temperature with 100 μl of tetramethylbenzidine-H₂O₂ substrate (Kirkegaard & Perry Laboratories, Gaithersburg, Md.) per well and the absorbance at 600 nm was measured.

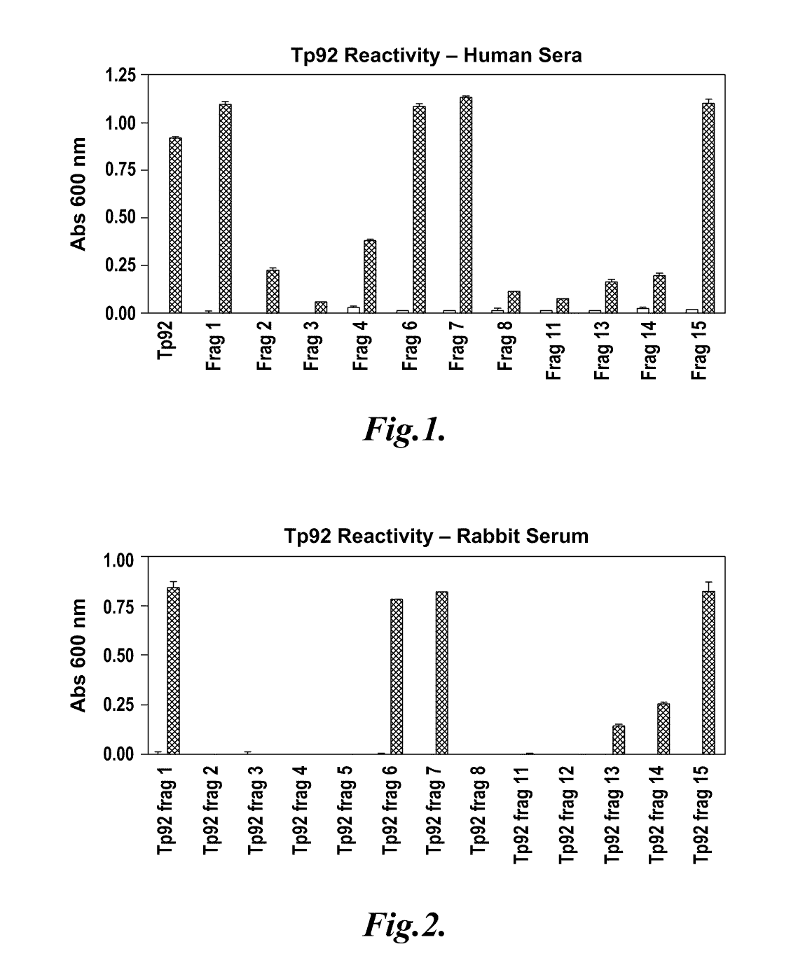

The results of these experiments are shown in FIG. 1. Additionally, similar studies were performed using serum collected from rabbits that were experimentally infected with T. pallidum. The rabbits were infected intratesticularly with 10⁵ Treponema pallidum subspecies pallidum and serum was collected at various time points after infection. The results of these experiments are shown in FIG. 2.

These experiments showed that the focus of the antibody response against T. pallidum infection is localized to a region within recombinant fragment 7 (amino acids 26-220) (SEQ ID NO:13).

### Example 2

This example describes the expression of polypeptide fragments of the Tp0453 protein and the identification of polypeptide fragments that bind antibodies from human syphilis patients.

To prepare recombinant proteins, coding sequences based on the ORFs for Tp92, TP0453, and Gpd were PCR amplified from T. pallidum subsp. pallidum (Nichols strain) genomic DNA with primers designed from the coding sequence of each gene. Proteins were expressed with the N-terminal signal sequence when applicable, except for Tp92-derived sequences. The full-length ORF of Gpd was also expressed. PCR products representing the nucleotide sequence encoding the polypeptide fragments were ligated in frame with expression plasmids. Nucleotide sequences from Tp92 and TP0453 were cloned using the pRSET T7 expression plasmid. Full-length Tp0453 protein and recombinant peptide fragments of Tp0453 were expressed and purified, as described in Example 1. The amino-terminal Tp0453 peptide fragments did not include the first 29 amino acids, as these amino acids likely represent a signal sequence that is cleaved from the mature protein and, therefore, would probably not play a role in the immunogenicity of Tp0453.

Human syphilis patient sera were obtained as frozen stocks from two sources—the collection of the Ludwig Boltzmann Institute for Dermato-Venerological Serodiagnostics, Vienna, Austria; and from the Marra laboratory at Harborview Medical Center, Seattle, Wash. Syphilis diagnosis for each of the sera were defined as follows: primary, typical chancre present; secondary, generalized typical rash and positive syphilis serology test; latent, positive syphilis serology tests and a positive T. pallidum particle agglutination assay (TPPA) or immunoglobulin M test to T. pallidum antigens (Mercia Syphilis M™ enzyme-linked immunosorbent assay [ELISA]; Microgen Bioproducts, Camberley, United Kingdom) with no history of treatment and no clinical manifestations; neurosyphilis, reactive cerebrospinal fluid (CSF) values plus TPHA index values greater than 70. The sera from the Bolzmann Institute were also tested for syphilis serology titers by the VDRL (Dade Behring, Marburg, Germany), the MHA-TP (Fujirebio, Tokyo, Japan), FTA-Abs, and Captia Syphilis-G™ enzyme immunoassay (EIA) (Ross, J., et al., Genitourin Med. 67:408-410, 1991) tests, whereas syphilis serology for the samples from Harborview Medical Center was determined by rapid plasma regain (RPR) test and TPPA. Eleven of the patients had been treated prior to collection of sera.

Sera from individuals with relapsing fever were obtained from the Centers for Disease Control and Prevention, Fort Collins, Colo., and from the collection of Rocky Mountain Laboratories, Hamilton, Mont. The criteria for relapsing fever diagnosis were clinical features of the disease, exposure to tick-borne relapsing fever in eastern Washington or northern Idaho, seropositivity greater than 1:2,048 in the Western blot for Borrelia hermsii HS1. Lyme disease patient sera were obtained from the Centers for Disease Control and Prevention collection (Fort Collins, Colo.). The criteria for the diagnosis of Lyme disease were residence in an area of endemicity, clinical manifestations consistent with Lyme disease, and more than five reactive bands by Western blot analysis of Borrelia burgdorferi. Sera from individuals with severe leptospirosis were obtained from the collection of the Oswaldo Cruz Foundation (Salvador, Bahia, Brazil). Criteria for diagnosis of the convalescent phase of leptospirosis was clinical history consistent with leptospirosis and laboratory-confirmed diagnosis according to microagglutination test criteria of a four-fold rise in agglutination titers or a reciprocal agglutination titer greater than 1:800. Convalescent-phase sera were obtained 14 to 28 days after hospitalization of these individuals for leptospirosis. In the city of Salvador, the etiologic agent is Leptospira interrogans serovar Copenhageni. Sera from uninfected controls were obtained from healthy volunteers in Seattle, Wash.

Elisa assays were performed as in Example 1. For experiments that included multiple different antigens (peptide fragments) per well, each antigen was coated at 2 μg/ml. Plates were decanted then blocked with 1×PBS-4% milk at room temperature for 2.5 hours. Human sera were diluted 1:100 in 1×PBS-4% milk-0.2% Triton X-100. For serum titration experiments, the human sera were serially diluted 1:2 from a maximum concentration of 1:100 to a minimal concentration of 1:3200. The diluted sera were adsorbed overnight at 4° C. with a 0.5% (vol/vol) lysate of E. coli expressing an irrelevant Trypanosoma cruzi recombinant protein (SA85-1.1) in pRSET, as described in Example 1.

In order to assign an individual test result as positive or negative for syphilis infection, the cutoff absorbance values for each recombinant antigen were defined as the mean plus three times the standard deviation of the absorbance of the uninfected sera; sera that gave absorbance values above the cutoff were considered positive, while those below the cutoff were considered negative. The cutoff values were calculated from the values obtained from the 12 uninfected control sera for each antigen or combination of antigens. The cutoff values for the recombinant protein ELISAs were as follows: 0.127 for Gpd.1; 0.070 for 453.2; 0.133 for Tp92.F7; 0.086 for the Tp92.F7/453.2 combination; and 0.071 for the Tp92.F7/453.2/Gpd.1 combination. Differences between groups were measured by chi-square analysis, and significance was set as P<0.05.

Full-length Tp0453 and recombinant peptide fragments of Tp0453 were expressed and purified as described in Example 1. The full-length T. pallidum Tp0453 protein consists of 287 amino acids (SEQ ID NO:4). Six recombinant protein fragments (SEQ ID NOs:22-27) were created that span the entire length of the Tp0453 protein (SEQ ID NO:4). Table 2 shows the amino acid boundaries of the peptide fragments (numbering is from the first amino acid of SEQ ID NO:4).

TABLE 2

| Fragment | Number of First and Last | |
|---|---|---|
| Number | Amino Acid Residue | SEQ ID NO: |
| Tp0453.1 | 30-168 | 22 |
| Tp0453.2 | 149-287 | 23 |
| Tp0453.3 | 30-108 | 24 |
| Tp0453.4 | 90-168 | 25 |
| Tp0453.5 | 149-228 | 26 |
| Tp0453.6 | 209-287 | 27 |

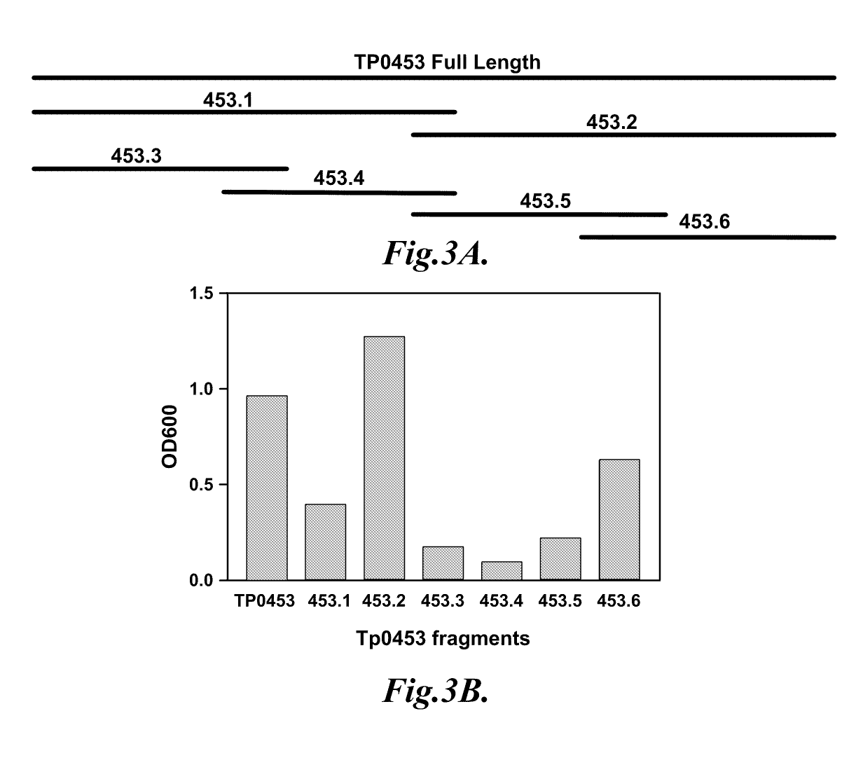

Elisa assays were conducted as described in Example 1 using the pooled serum samples described above. As shown in FIGS. 3A and 3B, full-length Tp0453 protein (SEQ ID NO:4), and the peptide fragments Tp0453.2 (SEQ ID NO:23), and Tp0453.6 (SEQ ID NO:27) bind antibodies present in sera pooled from humans at all stages of T. pallidum infection (1:100 dilution). The differential binding of antibodies from sera by Tp0453.2 (SEQ ID NO:23) and Tp0453.6 (SEQ ID NO:27) suggests that the immunodominant regions of Tp0453 contain multiple epitopes.

Accordingly, cross-absorption experiments were performed to determine which regions of the Tp0453.2 (SEQ ID NO:23) fragment contained epitopes that bind serum antibodies from humans infected with T. pallidum. For cross-absorption experiments, the absorbing peptide was added to the overnight incubation of pooled sera (diluted 1:100 in dilution buffer) at 50 ug/ml or 100 ug/ml. Plates were decanted and washed with TBST (45 mM Tris-HCl, 0.15 M NaCl, 0.5% v/v Tween 20). The remaining steps of the Elisa assay were as described in Example 1.

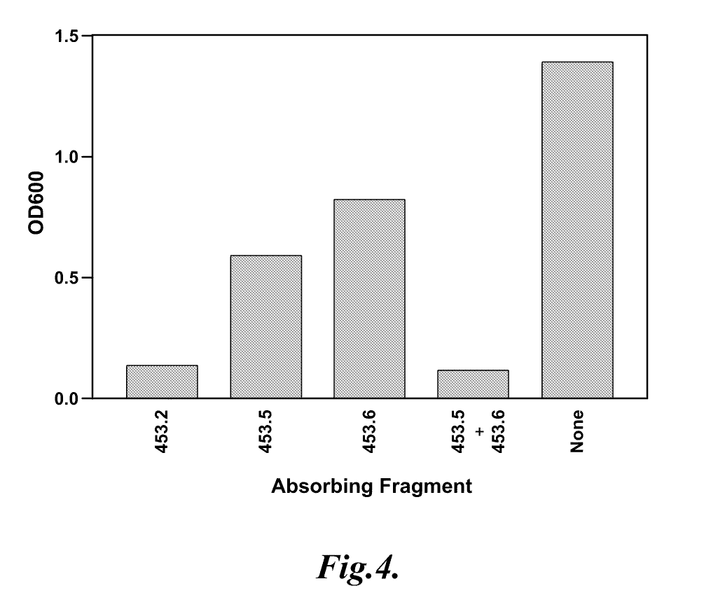

FIG. 4 shows the results of the cross-absorption experiment using pooled syphilis sera that were pre-absorbed with the indicated Tp0453 peptide fragments and then added to triplicate wells coated with the Tp0453.2 (SEQ ID NO:23) fragment and incubated for one hour at room temperature. As shown in FIG. 4, pre-absorption with the Tp0453.2 (SEQ ID NO:23) fragment removed nearly all of the binding activity to itself, whereas pre-absorption with Tp0453.5 (SEQ ID NO:26) or Tp0453.6 (SEQ ID NO:27) only partially removed antibody binding to Tp0453.2 (SEQ ID NO:23). Pre-absorption with Tp0453.5 (SEQ ID NO:26) plus Tp0453.6 (SEQ ID NO:27) in combination removed nearly all of the binding activity to Tp0453.2 (SEQ ID NO:23). This result demonstrates that multiple binding epitopes exist in the Tp0543.2 fragment.

In summary, this example shows that the region comprising amino acids 103 to 287 of the Tp0543 protein (SEQ ID NO:4) contains binding epitopes to antibodies in sera from human subjects infected with T. pallidum.

### Example 3

This example describes the expression of polypeptide fragments of the Gpd protein and the identification of polypeptide fragments that bind antibodies from human syphilis patients.

The full-length T. pallidum Gpd protein consists of 356 amino acids (SEQ ID NO:6). Sequences from Gpd were cloned using the BG 1861 vector (that expresses proteins as N-terminal His₆-ORF fusion proteins) and expressed in the E. coli expression strain BL21(DE3)/pLysS (Invitrogen, Carlsbad, Calif.). Full-length Gpd protein and recombinant peptide fragments of Gpd were expressed and purified as described in Example 1. Six recombinant protein fragments (SEQ ID NOs:28-33) were created that span the entire length of the Gpd protein (SEQ ID NO:6). Table 3 shows the amino acid boundaries of the peptide fragments (numbering is from the first amino acid of SEQ ID NO:6).

TABLE 3

| Fragment | Number of First and Last | |
|---|---|---|
| Number | Amino Acid Residue | SEQ ID NO: |
| Gpd.1 | 1-190 | 28 |
| Gpd.2 | 165-356 | 29 |
| Gpd.3 | 1-102 | 30 |
| Gpd.4 | 77-190 | 31 |
| Gpd.5 | 165-261 | 32 |
| Gpd.6 | 254-356 | 33 |

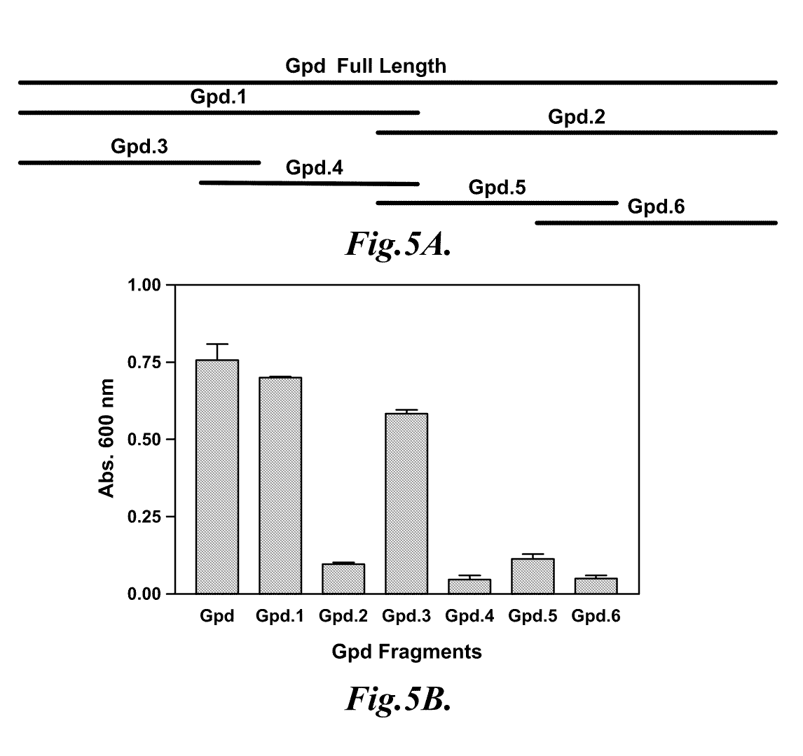

A pool of sera from untreated persons with syphilis was allowed to react with the fragments and a pool of uninfected human serum was used as a negative control. As shown in FIGS. 5A and 5B, full-length Gpd protein (SEQ ID NO:6) and the peptide fragments Gpd.1 (SEQ ID NO:28) and Gpd.3 (SEQ ID NO:30) bind antibodies present in sera pooled from humans at all stages of T. pallidum infection (1:100 dilution), indicating that the first 190 amino acids of the Gpd protein were highly immunoreactive.

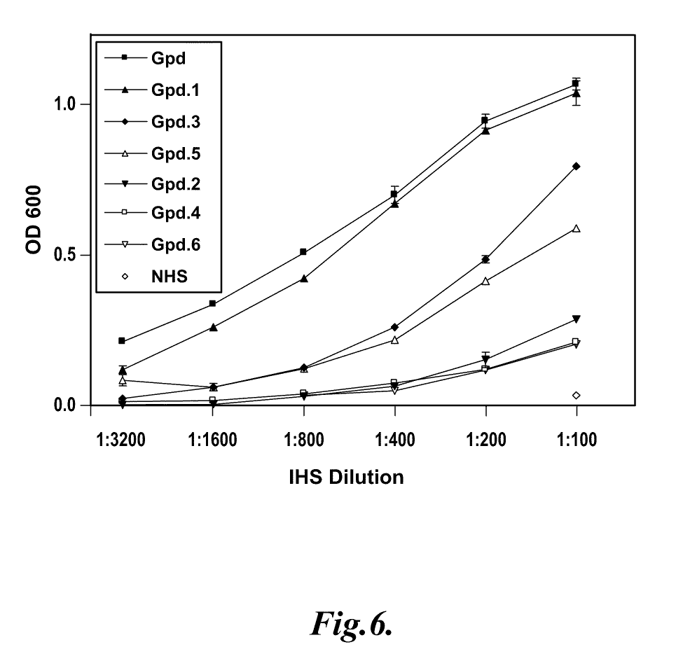

FIG. 6 shows serial dilution curves for full-length Gpd and each Gpd fragment. Although both fragments Gpd.1 (SEQ ID NO:28) and Gpd.3 (SEQ ID NO:30) reacted strongly at a single concentration of serum (see FIG. 5B) when serum was titrated against both fragments, Gpd.3 required approximately four times the serum concentration to achieve the same optical densities as Gpd.1 (FIG. 6). This observation likely indicates that Gpd.1 (SEQ ID NO:28) binds more antibodies than Gpd.3 (SEQ ID NO:30); though another possible explanation is that Gpd.1 binds the plate better than Gpd.3. The titration curve for Gpd.1 (SEQ ID NO:28) was only slightly below that for full-length Gpd (SEQ ID NO:6), indicating that most of the seroreactivity to Gpd is due to epitopes found on Gpd. 1.

This example shows that a 190 amino acid peptide N-terminal fragment of Gpd contains the dominant epitopes that bind antibodies present in sera from humans infected with T. pallidum.

### Example 4

This example shows that the peptide fragments identified above are useful and show high sensitivity in the serodiagnosis of various stages of syphilis in individual human subjects. The peptide fragments show high sensitivity when used individually, in combination, or in mixtures.

ELISA assays were performed as described in Examples 1-3 above. In order to assess each peptide fragment's utility for serodiagnosis, the fragments were tested individually with a panel of sera from human subjects at different stages of syphilis infection. Further, instead of testing a pool of sera, each serum was tested individually. A positive reaction was defined as measurements exceeding the mean plus three times the standard deviation of the OD obtained from 12 control seronegative human subjects. The immunodominant fragments Gpd.1 (SEQ ID NO:28), Tp0453.2 (SEQ ID NO:23), and Tp92 fragment F7 (SEQ ID NO:13) were chosen for analysis.

As shown in Table 4, each of the three fragments alone correctly identified greater than 80% of human subjects as infected with T. pallidum at each stage of infection. Further, each individual serum was positive with at least one of the three antigens (“At least 1 of 3” in Table 4), such that the combined results in each stage was 100% positive. In other words, the use of all three antigens separately yielded 100% sensitivity in the ability to detect true positives (human subjects infected with T. pallidum) for each stage of infection.

TABLE 4
SENSITIVITY OF INDIVIDUAL PEPTIDE FRAGMENTS.

| | Fragment | | | |
|---|---|---|---|---|
| Stage of | Gpd.1 (SEQ | 453.2 (SEQ | Tp92.F7 (SEQ | At least 1 |
| Infection | ID NO: 28) | ID NO: 23) | ID NO: 13) | of 3 |
| Primary, | 83% | 92% | 92% | 100% |
| n = 12 | | | | |
| Secondary, | 100% | 100% | 100% | 100% |
| n = 15 | | | | |
| Late Latent, | 88% | 88% | 100% | 100% |
| n = 8 | | | | |
| Neurosyphilis, | 100% | 100% | 100% | 100% |
| n = 6 | | | | |

Table 5 shows that the sensitivity can be further increased by using mixtures of combinations of the immunodominant peptide fragments from each protein. The indicated combinations of peptide fragments were mixed and coated in each ELISA well, and ELISA assays performed as in Example 2. Serum from individual human subjects at different stages of infection was tested separately. The combination of Tp92.F7 (SEQ ID NO:13) and Tp453.2 (SEQ ID NO:23) gave an overall sensitivity of 92%, while the combination of three antigens Tp92.F7 (SEQ ID NO:13), Tp453.2 (SEQ ID NO:23), and Gpd.1 (SEQ ID NO:28) mixed and coated in each ELISA well gave an overall sensitivity of 95% (p value=0.033).

TABLE 5
SENSITIVITY OF PEPTIDE FRAGMENT COMBINATIONS.

| | Fragment Combinations | |
|---|---|---|
| | | Tp92.F7 (SEQ ID |
| | Tp92.F7 (SEQ ID | NO: 13) and 453.2 |
| | NO: 13) and 453.2 | (SEQ ID NO: 23) and |
| Stage of Infection | (SEQ ID NO: 23) | Gpd.1 (SEQ ID NO: 28) |
| Primary, n = 47 | 92% | 96% |
| Secondary, n = 98 | 97% | 96% |
| Latent, n = 120 | 88% | 95% |
| Neurosyphilis, n = 28 | 92% | 95% |
| All, n = 293 | 92% | 95% |

As shown in Table 5, there was a statistically significant increase in the number of positive sera detected using three versus two antigens. The majority of false-negatives were obtained using sera from subjects with latent syphilis. Unlike most serodiagnostic tests—for example, those described in Manavi, K., et al., Int. J. STD AIDS 17:768-771, 2006—the assay described in Table 5 performed as well when using sera from primary syphilis stages as when using sera from any other stage of disease.

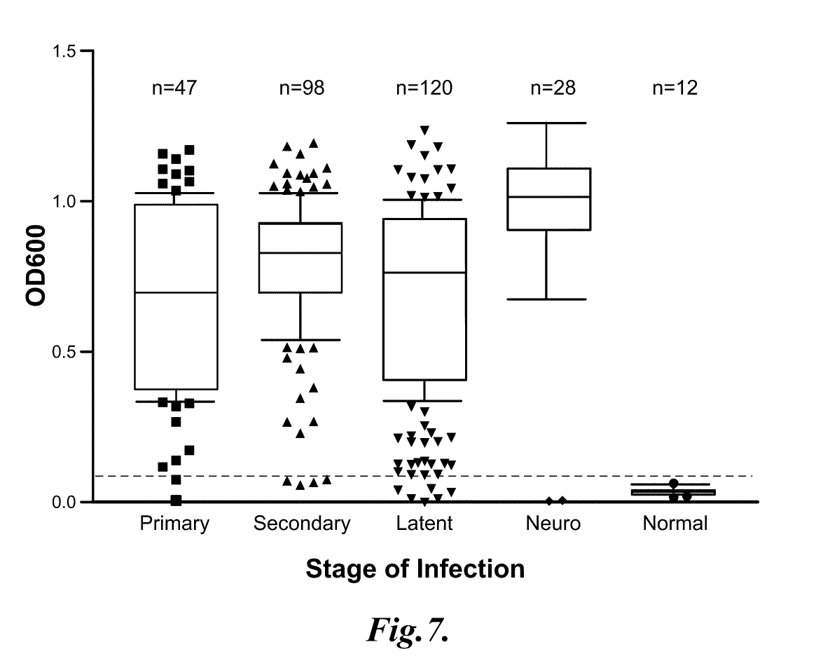

FIG. 7 shows the mean absorbance values obtained for sera from human subjects at various stages of syphilis infection in ELISA using the combination of peptide fragment antigens Tp92.F7 (SEQ ID NO:13), Tp453.2 (SEQ ID NO:23), and Gpd.1 (SEQ ID NO:28). The stages tested were primary, secondary, latent, and neurological infection stages.

In summary, this example demonstrates that using a combination of three immunodominant peptide fragments, one each from Tp92, Tp0453, and Gpd proteins, achieved a high level of sensitivity in diagnosing individual human subjects infected with syphilis.

### Example 5

This example shows that using combinations of peptide fragments is highly specific for the serodiagnosis of human subjects infected with syphilis.

Example 4 demonstrated that the ELISA was more sensitive when using a combination of three peptide fragment antigens coated on the same well rather than a combination of Tp92.F7 (SEQ ID NO:13) and Tp453.2 (SEQ ID NO:23). To determine if this increased sensitivity could be achieved without drastically decreasing the specificity of the test, a panel of sera from individual human subjects with non-syphilis spirochetal infections including Lyme disease, leptospirosis, relapsing fever, and periodontal disease was tested. Specificity refers to the ability of the test to exclude those subjects who were not infected with T. pallidum. The ELISA was performed by coating the wells with either 2 antigens or 3 antigens in combination and running them against individual control sera. Positives are defined as those greater than the mean of uninfected controls plus 3 times the standard deviation of the mean. The specificity was determined by subtracting the percentage of positive sera for each disease from 100%. For example, 0% of the sera from uninfected subjects was positive; therefore, the specificity of the test was 100%.

As shown in Table 6, the specificities for the combination of Tp92.F7 (SEQ ID NO:13) and Tp453.2 (SEQ ID NO:23) and the combination of three antigens Tp92.F7 (SEQ ID NO:13), Tp453.2 (SEQ ID NO:23), and Gpd.1 (SEQ ID NO:28) were identical, that is 96%. Thus, inclusion of Gpd.1 (SEQ ID NO:28) in the antigen combination increases sensitivity apparently without decreasing specificity.

TABLE 6
SPECIFICITY OF PEPTIDE FRAGMENT
COMBINATION ELISA TESTS.

| | Fragment Combinations | |
|---|---|---|
| | | 453.2 (SEQ ID |
| | 453.2 (SEQ ID | NO: 23) + Tp92.F7 |
| Non-syphilis | NO: 23) + Tp92.F7 | (SEQ ID NO: 13) + |
| Disease | (SEQ ID NO: 13) | Gpd.1 (SEQ ID NO: 28) |
| Uninfected, | 100% | 100% |
| n = 12 | | |
| Leptospirosis, | 94% | 94% |
| n = 18 | | |
| Relapsing | 100% | 100% |
| fever, n = 8 | | |
| Lyme disease, n = 15 | 93% | 93% |
| Periodontal | 95% | 95% |
| disease, | | |
| n = 21 | | |
| All, n = 74 | 96% | 96% |

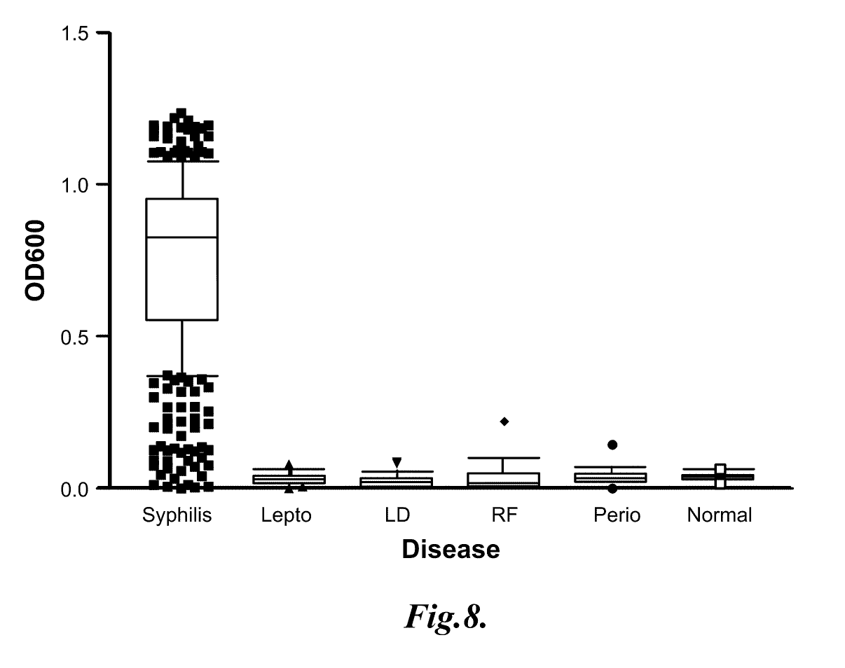

FIG. 8 shows the mean absorbance values for sera from human subjects having syphilis, leptospirosis, Lyme disease, relapsing fever, periodontal disease, and uninfected controls (normal) in ELISA using the combination of peptide fragment antigens Tp92.F7 (SEQ ID NO:13), Tp453.2 (SEQ ID NO:23), and Gpd.1 (SEQ ID NO:28).

Table 7 summarizes the results using a combination of the three peptide fragments Tp92.F7 (SEQ ID NO:13), Tp453.2 (SEQ ID NO:23), and Gpd.1 (SEQ ID NO:28) in diagnostic tests regarding sensitivity, specificity, positive predictive value, and negative predictive value. Positive predictive value refers to the ability of the test to predict which human subjects are infected, whereas negative predictive value refers to the ability of the test to predict which human subjects are not infected with the disease.

TABLE 7
EFFICACY OF THE COMBINATION ELISA TEST.

| | |
|---|---|
| Overall sensitivity | 95% |
| Overall specificity | 96% |
| Positive predictive value | 99% |
| Negative predictive value | 75% |

In summary, this example demonstrates that the combination of peptide fragments Tp92.F7 (SEQ ID NO:13), Tp453.2 (SEQ ID NO:23), and Gpd.1 (SEQ ID NO:28) is able to detect antibodies from human subjects infected with T. pallidum at a high level of specificity.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for specific detection of antibodies resulting from exposure to or infection with Treponema pallidum in a biological sample from a test subject, comprising the steps of:
(a) contacting the biological sample with one or more isolated polypeptides having a sequence at least 95% identical to a polypeptide selected from the group consisting of:
(i) a polypeptide fragment of a Tp92 protein having no more than 740 amino acids, the fragment having the sequence of SEQ ID NO:12 or an immunogenic fragment thereof;
(ii) a polypeptide fragment of a Tp0453 protein having no more than 140 amino acids, the fragment having the sequence of SEQ ID NO:23 or an immunogenic fragment thereof; and
(iii) a polypeptide fragment of a Gpd protein having no more than 195 amino acids, the fragment having the sequence of SEQ ID NO:28 or an immunogenic fragment thereof; and
(b) detecting whether antigen-antibody binding has occurred between the polypeptide and an antibody component of the biological sample, in which the detection of antigen-antibody binding indicates the presence of antibodies in the biological sample and is indicative of exposure to or infection of the test subject with Treponema pallidum.

2. The method of claim 1, wherein the immunogenic polypeptide fragment of a Tp92 protein having no more than 740 amino acids comprises SEQ ID NO:7, SEQ ID NO:13, or SEQ ID NO:21.

3. The method of claim 1, wherein the immunogenic polypeptide fragment of a Tp92 protein having no more than 740 amino acids comprises SEQ ID NO:13.

4. The method of claim 1, wherein the immunogenic polypeptide fragment of a Tp0453 protein having no more than 140 amino acids comprises SEQ ID NO:26 or SEQ ID NO:27.

5. The method of claim 1, wherein the immunogenic polypeptide fragment of a Gpd protein having no more than 195 amino acids comprises SEQ ID NO:30.

6. The method of claim 1, wherein a combination or mixture of two or more isolated polypeptides is contacted with the biological sample.

7. The method of claim 6, wherein the isolated polypeptides are separately contacted with the biological sample.

8. The method of claim 6, wherein the isolated polypeptides are covalently linked to each other.

9. The method of claim 1, wherein the bodily fluid is selected from the group consisting of whole blood, blood serum, blood plasma, and saliva.

10. The method of claim 1, wherein antigen-antibody binding is detected using a labeled anti-class-specific antibody or anti-subclass specific antibody.

11. The method of claim 10, wherein the anti-class-specific antibody is selected from the group consisting of an anti-IgM antibody, an anti-IgG antibody, and an anti-IgA antibody.

12. The method of claim 10, wherein the anti-subclass-specific antibody is selected from the group consisting of an anti-IgG1 antibody, an anti-IgG2a antibody, an anti-IgG2b antibody, an anti-IgG3 antibody and an anti-IgG4 antibody.

13. The method of claim 1, wherein the one or more isolated polypeptides are bound to a substrate.

14. A method for specific detection of antibodies resulting from exposure to or infection with Treponema pallidum in a biological sample from a test subject, comprising the steps of:
(a) contacting the biological sample with one or more isolated polypeptides having no more than 200 amino acids and a sequence at least 95% identical to a polypeptide selected from the group consisting of:
(i) a polypeptide fragment of a Tp92 protein having the sequence of SEQ ID NO:13 or an immunogenic fragment thereof;
(ii) a polypeptide fragment of a Tp0453 protein having the sequence of SEQ ID NO:23 or an immunogenic fragment thereof; and
(iii) a polypeptide fragment of a Gpd protein having the sequence of SEQ ID NO:28 or an immunogenic fragment thereof; and
(b) detecting whether antigen-antibody binding has occurred between the polypeptide and an antibody component of the biological sample, in which the detection of antigen-antibody binding indicates the presence of antibodies in the biological sample and is indicative of exposure to or infection of the test subject with Treponema pallidum.

15. A kit comprising:
(a) one or more isolated polypeptides having a sequence at least 95% identical to a polypeptide selected from the group consisting of:
(i) a polypeptide fragment of a Tp92 protein having no more than 740 amino acids, the fragment having the sequence of SEQ ID NO:12 or an immunogenic fragment thereof;
(ii) a polypeptide fragment of a Tp0453 protein having no more than 140 amino acids, the fragment having the sequence of SEQ ID NO:23 or an immunogenic fragment thereof; and
(iii) a polypeptide fragment of a Gpd protein having no more than 195 amino acids, the fragment having the sequence of SEQ ID NO:28 or an immunogenic fragment thereof;
(b) reagents for labeling antibody bound to the isolated polypeptide; and
(c) written indicia providing a user with instructions for use of the kit to determine whether a human subject is infected with T. pallidum.

16. An isolated polypeptide comprising a sequence at least 95% identical to a polypeptide selected from the group consisting of:
(i) a polypeptide fragment of a Tp92 protein having no more than 740 amino acids, the fragment having the sequence of SEQ ID NO:12 or an immunogenic fragment thereof;
(ii) a polypeptide fragment of a Tp0453 protein having no more than 140 amino acids, the fragment having the sequence of SEQ ID NO:23 or an immunogenic fragment thereof; and
(iii) a polypeptide fragment of a Gpd protein having no more than 195 amino acids, the fragment having the sequence of SEQ ID NO:28 or an immunogenic fragment thereof.

17. An expression vector comprising a nucleic acid sequence that encodes a polypeptide comprising a sequence at least 95% identical to a polypeptide selected from the group consisting of:
(i) a polypeptide fragment of a Tp92 protein having no more than 740 amino acids, the fragment having the sequence of SEQ ID NO:12 or an immunogenic fragment thereof;
(ii) a polypeptide fragment of a Tp0453 protein having no more than 140 amino acids, the fragment having the sequence of SEQ ID NO:23 or an immunogenic fragment thereof; and
(iii) a polypeptide fragment of a Gpd protein having no more than 195 amino acids, the fragment having the sequence of SEQ ID NO:28 or an immunogenic fragment thereof.

18. A living cell that comprises the expression vector of claim 16.


SEQUENCE LISTING (33 sequences)

SEQ ID NO: 1 - DNA, 2514 bp, Treponema Pallidum
  CDS (1)..(2514)
atgctcaaaaaagccagtgccttcctaattgcaagttgttgtgtgatgtcgctggcgtgggcacaggcaaacgacaattggtacgagggaaagcctatctctgcgattagttttgaggggctcgaatatattgctcgcggccagttggacacgattttttctcaatacaagggacaaaagtggacctatgagctgtacctggagatactgcaaaaggtctatgaccttgagtacttttctgaagtttcgcctaaggcggtgcccaccgatccggagtatcagtatgtgatgctacagttcacggtaaaggagcgtccttcggtgaagggcatcaagatggtagggaacagccaaatccgcagtggggaccttttgtctaaaatcctcctgaaaaagggagacatttacaatgaagtaaagatgaaggtggaccaagagtcgctcaggcgtcattacctggaccagggctatgcggcggttaagatatcctgcgaggcaaaaactgaggcggggggcgtggtggtacagtttaccatccaggaaggtaagcagactgttgtctcgcggatacagtttaagggaaataaggcgtttaccgagtcggtgctcaagaaggtgctttccacgcaggaggcgcgttttttgaccagtggggtgttcaaggagaatgcgctggaagcggataaggcggcagtccactcatactatgcagagaggggatacattgacgcgcgggtagaaggcgtggcaaagacggttgataaaaaaactgacgccagtcgcaatctggttacgcttacgtacactgtggtggaaggtgagcagtaccgctacggcggggttaccattgtgggtaaccagatttttagcaccgaggagctgcaggcaaaaattaggctcaagcgcggggccatcatgaatatggtggcctttgagcagggctttcaggcgctggcggatgcgtattttgaaaacggatacacgtcaaattacctgaacaaagaagaacaccgggacacggcggagaaaacgctttcgtttaagatcacggtggtggagcgcgagcgcagccacgtcgagcacattatcattaagggaacgaagaatacaaaagacgaggttatcctgcgtgaaatgctgctgaaaccgggggatgtgttctctaagtcaaagtttacggatagcttgcgcaatctgttcaacctgcgctatttctcgtcgctggtgccggatgtgcggcccggctctgagcaggacctggtggacattatcctgaatgtggaggagcagtcgacggcaaacgtgcagtttggggtgacgttttctggggtgggggaggcaggcacgttcccgctttcgctcttttgtcagtgggaagaaaagaattttttgggaaaagggaatgaaatttcagtaaatgcaaccttggggtctgaggcgcagagcctgaagctcgggtatgtggagcgctggtttctgggctctccgctgacggtgggctttgactttgaacttacgcacaaaaatctctttgtgtaccgcgcgggttcatacggcaacgggctgccgcacccgtacacgagcagggagcagtgggctagttcccctgggctggcagaatcgtttcgcctcaagtattcgcgctttgagtccgccatcggcgcgcacaccgggtaccagtggtatccgcgctatgcggtcattagggtgaacgggggggtggactttcgggttgtaaagaatttttacgataaggataacaatcagcccttcgacctgaccgtaaaagagcagctgaactggaccagtatcaattcgttttggacgagcgtttcgtttgacgggcgtgactttgcgtacgacccgtccagcggctggtttttaggacagcgctgtacgttcaacgggctcgttccctttctcgaaaaagagcattcgtttcgctccgacaccaaggccgagttctacgttaccctgctcaattatccggtctctgccgtgtggaacttaaagtttgtcttggctttctacaccggtgtgtccgttcaaacgtattatggacggaggaaaagcgaaaacggaaagggcaacggggtgcggtccggcgcgctggtaatagacggcgtgctggtagggcgcgggtggagcgaagacgcaaagaaaaacaccggagacctgctgctccaccactggattgagttccgctggccgctggcgcacggcattgtgtcctttgactttttctttgatgcggcaatggtgtacaacatcgaaagtcagtccccaaacgggtcatcgtccgccagcagctccagcagcagcagtagtagtagcagtagaaccaccagctctgaaggactgtacaaaatgagctacggtccggggctgcgctttacattgccgcaatttccgttaaaattggcgttcgcaaacaccttcacgtcacccggcggcatcccaaaaacaaagaaaaattggaattttgtgttgtcgttcacggtaaataatttgtag

SEQ ID NO: 2 - protein, 837 aa, Treponema Pallidum
MLKKASAFLIASCCVMSLAWAQANDNWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFDLTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNGSSSASSSSSSSSSSSRTTSSEGLYKMSYGPGLRFTLPQFPLKLAFANTFTSPGGIPKTKKNWNFVLSFTVNNL

SEQ ID NO: 3 - DNA, 864 bp, Treponema Pallidum
  CDS (1)..(864)
ttgataaggcgtagatatcgtggttgtacgcagggagcgtggatagtaagtgttggtatgctatttgcatcgtgcacttcaggggcgtggaaggcatcagtagatccgttgggggttgtgggatctggtgcagatgtgtacctgtatttccctgtagcggggaacgagaatttgatttctcgtattatcgagaaccatgagtcaaaggcagatattaaaaaaatagtggacaggactaccgcggtatacggtgctttttttgcccgatcaaaagagtttcgtttgttcggaagcggttcgtatccatacgcctttactaatttgattttttctcgatccgatggctgggcatctacgaaaacggaacacggaatcacgtactatgaaagtgaacatacggacgtttcgattcctgcgccgcatttttcctgtgtgatttttggttcctccaaaagggagcggatgagcaaaatgctgtctcggctcgttaaccccgatcgaccgcagttaccgcctcgctttgaaaaagaatgtacgtcggaaggtacgagccagactgttgcactctatataaaaaacgggggacactttattaccaaactgttgaattttccgcagcttaatttaccacttggggcaatggaactgtacttgaccgcgcggaggaatgagtatctttacacgttgagcttgcagctggggaatgcaaagataaattttcccatacagtttttaatctctcgtgtgcttaacgcgcacattcacgtggagggggacaggttaattattgaagacggcacaatttctgctgagcgtcttgcgtcggtgatctcgtcgttgtactccaaaaagggaagttcgtaa

SEQ ID NO: 4 - protein, 287 aa, Treponema Pallidum
LIRRRYRGCTQGAWIVSVGMLFASCTSGAWKASVDPLGVVGSGADVYLYFPVAGNENLISRIIENHESKADIKKIVDRTTAVYGAFFARSKEFRLFGSGSYPYAFTNLIFSRSDGWASTKTEHGITYYESEHTDVSIPAPHFSCVIFGSSKRERMSKMLSRLVNPDRPQLPPRFEKECTSEGTSQTVALYIKNGGHFITKLLNFPQLNLPLGAMELYLTARRNEYLYTLSLQLGNAKINFPIQFLISRVLNAHIHVEGDRLIIEDGTISAERLASVISSLYSKKGSS

SEQ ID NO: 5 - DNA, 1071 bp, Treponema Pallidum
  CDS (1)..(1071)
atgcggggaacatattgtgtgacgctttgggggggggtgtttgcggcattggttgcaggctgtgcgtccgaacgtatgatagttgcgtatcggggcgctgcaggatatgtgcccgagcacacctttgcctcgaaagttcttgcttttgcacaaggagcagattacctgcagcaggatgtcgtgctttcaaaggataatcagcttatcgtagcgcaaagccatattctggataatatgactgacgtggcagaaaaatttccacgccggcagcgtgcggatgggcatttctatgtcatagattttacggtagaagaactttccctcctccgtgcaaccaatagtttctatacgcgcggtaagcgacatacgccggtgtatggccagcgctttcctctttggaagcctggttttaggctgcacacttttgaagaggagttgcagtttatccgtgggttggaacagacaaccgggaaaaagattggaatttactctgaaataaaggtgccgtggtttcatcatcaggaaggaaaagacatcgcagcgcttaccctcgctctgttgaaaaaatacggttaccaaagtcgatcggatctagtgtatgtgcaaacgtatgattttaacgagctgaagcgtatcaaacgagaacttttaccaaagtacgaaatgaacgtgaagctgattcagcgtgttgcttacacagatcaacgtgaaacacaggagaaggactcgcgtgggaaatggataaactacaattacaattggatgtttgagcccggtggtatgcagaaaatagcaaaatatgcagacggcgtgggtcctgactggaggatgctcatagagaatgaatggtcgaaggtgggcgctgttcgcctgagtccgatggtttctgcaatccaagatgcgaaattggaatgtcatgtgcacacggtacggaaagaaacactgcctagctacgcgcgcaccatggacgagatgttttccattttgttcaaacagacgggcgcaaacgtggtgctcacggattttcctgatcttggggtaaagtttctgggcaaacccgcccgctattga

SEQ ID NO: 6 - protein, 356 aa, Treponema Pallidum
MRGTYCVTLWGGVFAALVAGCASERMIVAYRGAAGYVPEHTFASKVLAFAQGADYLQQDVVLSKDNQLIVAQSHILDNMTDVAEKFPRRQRADGHFYVIDFTVEELSLLRATNSFYTRGKRHTPVYGQRFPLWKPGFRLHTFEEELQFIRGLEQTTGKKIGIYSEIKVPWFHHQEGKDIAALTLALLKKYGYQSRSDLVYVQTYDFNELKRIKRELLPKYEMNVKLIQRVAYTDQRETQEKDSRGKWINYNYNWMFEPGGMQKIAKYADGVGPDWRMLIENEWSKVGAVRLSPMVSAIQDAKLECHVHTVRKETLPSYARTMDEMFSILFKQTGANVVLTDFPDLGVKFLGKPARY

SEQ ID NO: 7 - protein, 567 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFD

SEQ ID NO: 8 - protein, 245 aa, Treponema Pallidum
LTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNGSSSASSSSSSSSSSSRTTSSEGLYKMSYGPGLRFTLPQFPLKLAFANTFTSPGGIPKTKKNWNFVLSFTVNNL

SEQ ID NO: 9 - protein, 167 aa, Treponema Pallidum
LTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIES

SEQ ID NO: 10 - protein, 89 aa, Treponema Pallidum
FDAAMVYNIESQSPNGSSSASSSSSSSSSSSRTTSSEGLYKMSYGPGLRFTLPQFPLKLAFANTFTSPGGIPKTKKNWNFVLSFTVNNL

SEQ ID NO: 11 - protein, 53 aa, Treponema Pallidum
EGLYKMSYGPGLRFTLPQFPLKLAFANTFTSPGGIPKTKKNWNFVLSFTVNNL

SEQ ID NO: 12 - protein, 739 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFDLTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNG

SEQ ID NO: 13 - protein, 195 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVF

SEQ ID NO: 14 - protein, 372 aa, Treponema Pallidum
KENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFD

SEQ ID NO: 15 - protein, 98 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGD

SEQ ID NO: 16 - protein, 97 aa, Treponema Pallidum
LLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVF

SEQ ID NO: 17 - protein, 124 aa, Treponema Pallidum
KENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKT

SEQ ID NO: 18 - protein, 112 aa, Treponema Pallidum
LSFKITVVERERSHVEHIIIKGTKNTKDEVILREMLLKPGDVFSKSKFTDSLRNLFNLRYFSSLVPDVRPGSEQDLVDIILNVEEQSTANVQFGVTFSGVGEAGTFPLSLFC

SEQ ID NO: 19 - protein, 136 aa, Treponema Pallidum
QWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFD

SEQ ID NO: 20 - protein, 372 aa, Treponema Pallidum
YFSSLVPDVRPGSEQDLVDIILNVEEQSTANVQFGVTFSGVGEAGTFPLSLFCQWEEKNFLGKGNEISVNATLGSEAQSLKLGYVERWFLGSPLTVGFDFELTHKNLFVYRAGSYGNGLPHPYTSREQWASSPGLAESFRLKYSRFESAIGAHTGYQWYPRYAVIRVNGGVDFRVVKNFYDKDNNQPFDLTVKEQLNWTSINSFWTSVSFDGRDFAYDPSSGWFLGQRCTFNGLVPFLEKEHSFRSDTKAEFYVTLLNYPVSAVWNLKFVLAFYTGVSVQTYYGRRKSENGKGNGVRSGALVIDGVLVGRGWSEDAKKNTGDLLLHHWIEFRWPLAHGIVSFDFFFDAAMVYNIESQSPNGSSSASSSSSSS

SEQ ID NO: 21 - protein, 378 aa, Treponema Pallidum
NWYEGKPISAISFEGLEYIARGQLDTIFSQYKGQKWTYELYLEILQKVYDLEYFSEVSPKAVPTDPEYQYVMLQFTVKERPSVKGIKMVGNSQIRSGDLLSKILLKKGDIYNEVKMKVDQESLRRHYLDQGYAAVKISCEAKTEAGGVVVQFTIQEGKQTVVSRIQFKGNKAFTESVLKKVLSTQEARFLTSGVFKENALEADKAAVHSYYAERGYIDARVEGVAKTVDKKTDASRNLVTLTYTVVEGEQYRYGGVTIVGNQIFSTEELQAKIRLKRGAIMNMVAFEQGFQALADAYFENGYTSNYLNKEEHRDTAEKTLSFKITVVERERSHVEHIIIKGTKNTKDEVILREMLLKPGDVFSKSKFTDSLRNLFNLR

SEQ ID NO: 22 - protein, 139 aa, Treponema Pallidum
WKASVDPLGVVGSGADVYLYFPVAGNENLISRIIENHESKADIKKIVDRTTAVYGAFFARSKEFRLFGSGSYPYAFTNLIFSRSDGWASTKTEHGITYYESEHTDVSIPAPHFSCVIFGSSKRERMSKMLSRLVNPDRP

SEQ ID NO: 23 - protein, 139 aa, Treponema Pallidum
SSKRERMSKMLSRLVNPDRPQLPPRFEKECTSEGTSQTVALYIKNGGHFITKLLNFPQLNLPLGAMELYLTARRNEYLYTLSLQLGNAKINFPIQFLISRVLNAHIHVEGDRLIIEDGTISAERLASVISSLYSKKGSS

SEQ ID NO: 24 - protein, 79 aa, Treponema Pallidum
WKASVDPLGVVGSGADVYLYFPVAGNENLISRIIENHESKADIKKIVDRTTAVYGAFFARSKEFRLFGSGSYPYAFTNL

SEQ ID NO: 25 - protein, 79 aa, Treponema Pallidum
SKEFRLFGSGSYPYAFTNLIFSRSDGWASTKTEHGITYYESEHTDVSIPAPHFSCVIFGSSKRERMSKMLSRLVNPDRP

SEQ ID NO: 26 - protein, 80 aa, Treponema Pallidum
SSKRERMSKMLSRLVNPDRPQLPPRFEKECTSEGTSQTVALYIKNGGHFITKLLNFPQLNLPLGAMELYLTARRNEYLYT

SEQ ID NO: 27 - protein, 79 aa, Treponema Pallidum
LPLGAMELYLTARRNEYLYTLSLQLGNAKINFPIQFLISRVLNAHIHVEGDRLIIEDGTISAERLASVISSLYSKKGSS

SEQ ID NO: 28 - protein, 190 aa, Treponema Pallidum
MRGTYCVTLWGGVFAALVAGCASERMIVAYRGAAGYVPEHTFASKVLAFAQGADYLQQDVVLSKDNQLIVAQSHILDNMTDVAEKFPRRQRADGHFYVIDFTVEELSLLRATNSFYTRGKRHTPVYGQRFPLWKPGFRLHTFEEELQFIRGLEQTTGKKIGIYSEIKVPWFHHQEGKDIAALTLALLKKY

SEQ ID NO: 29 - protein, 192 aa, Treponema Pallidum
EIKVPWFHHQEGKDIAALTLALLKKYGYQSRSDLVYVQTYDFNELKRIKRELLPKYEMNVKLIQRVAYTDQRETQEKDSRGKWINYNYNWMFEPGGMQKIAKYADGVGPDWRMLIENEWSKVGAVRLSPMVSAIQDAKLECHVHTVRKETLPSYARTMDEMFSILFKQTGANVVLTDFPDLGVKFLGKPARY

SEQ ID NO: 30 - protein, 102 aa, Treponema Pallidum
MRGTYCVTLWGGVFAALVAGCASERMIVAYRGAAGYVPEHTFASKVLAFAQGADYLQQDVVLSKDNQLIVAQSHILDNMTDVAEKFPRRQRADGHFYVIDFT

SEQ ID NO: 31 - protein, 114 aa, Treponema Pallidum
DNMTDVAEKFPRRQRADGHFYVIDFTVEELSLLRATNSFYTRGKRHTPVYGQRFPLWKPGFRLHTFEEELQFIRGLEQTTGKKIGIYSEIKVPWFHHQEGKDIAALTLALLKKY

SEQ ID NO: 32 - protein, 97 aa, Treponema Pallidum
EIKVPWFHHQEGKDIAALTLALLKKYGYQSRSDLVYVQTYDFNELKRIKRELLPKYEMNVKLIQRVAYTDQRETQEKDSRGKWINYNYNWMFEPGGM

SEQ ID NO: 33 - protein, 103 aa, Treponema Pallidum
WMFEPGGMQKIAKYADGVGPDWRMLIENEWSKVGAVRLSPMVSAIQDAKLECHVHTVRKETLPSYARTMDEMFSILFKQTGANVVLTDFPDLGVKFLGKPARY